(19) United States
(12) Patent Application Publication
(10) Pub. No.: US 2025/0236877 A1
(43) Pub. Date: Jul. 24, 2025

(54) HIGH-YIELD AND LARGE PANICLE GENE OLGN8.2 FROM WILD RICE ORYZA LONGISTAMINATA AND APPLICATIONS THEREOF

(71) Applicant: WUHAN UNIVERSITY, Hubei (CN)

(72) Inventors: Shaoqing LI, Hubei (CN); Manman LIU, Hubei (CN); Fengfeng FAN, Hubei (CN); Nengwu LI, Hubei (CN); Xianlong LI, Hubei (CN)

(73) Assignee: WUHAN UNIVERSITY, Hubei (CN)

(21) Appl. No.: 18/983,398

(22) Filed: Dec. 17, 2024

(30) Foreign Application Priority Data
Jan. 19, 2024 (CN) 202410078912.4

(51) Int. Cl. C12N 15/82; C07K 14/415

(52) U.S. Cl. CPC C12N 15/8261; C07K 14/415

(57) ABSTRACT

This invention discloses a new gene, OlGn8.2, derived from wild rice Oryza longistaminata and applications thereof, belonging to the field of crop genetic engineering and molecular biology techniques. The OlGn8.2 was introduced into the near-isogenic line NIL-OlGn8.2 or overexpressed in rice variety 9311, resulting in thicker stems and a significant increase in the number of primary and secondary branches as well as the number of grains per panicle. These results indicate that the OlGn8.2 significantly enhances rice yield by promoting growth and development, increasing the development of the primary and secondary branches, and consequently increasing the number of grains per panicle. Therefore, the OlGn8.2 has great potential for application in the breeding of high-yield rice varieties.

### CROSS-REFERENCE TO RELATED APPLICATION

This application claims the priority benefit of China application serial no. 202410078912.4, filed on Jan. 19, 2024. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Sep. 18, 2024, is named 148373-US-sequence listing and is 10,141 bytes in size.

### BACKGROUND

### Technical Field

This invention relates to crop genetic engineering and molecular biology, specifically a high-yield and large panicle gene OlGn8.2 from wild rice Oryza longistaminata and applications thereof.

### Description of Related Art

Rice, is the world's largest staple food crop, feeds more than half of the global population. With the rapid growth of the world's population and the deterioration of the global climate and environment, the supply gap for rice continues to widen. Therefore, continuously increasing rice yield and grain production is crucial to ensuring global food security in the 21st century. To improve rice yield, it is important to explore and use genes from rice or its wild relatives that are high yielding. An African wild rice species, Oryza longistaminata, possesses many excellent traits such as strong stems, lodging resistance, large panicles with plentiful grains, disease and pest resistance, and strong stress tolerance. It serves as an important gene resource pool for the genetic improvement of rice. Exploration and utilization of high-yielding and disease-resistant genes from Oryza longistaminata are significant for promoting high-yield improvement in rice and ensuring food security.

Rice yield is mainly determined by effective panicles, grains per panicle, grain setting rate, and thousand-grain weight. Cloning rice genes related to yield traits, and applying these excellent genes and their favorable alleles to rice breeding will undoubtedly lead to the cultivation of high-yield rice varieties, which will help alleviate the food shortage caused by the rapid growth of the population.

FHY3 (Far-red elongated hypocotyls 3) transcription factors constitute a gene family unique to plants. Several studies have demonstrated that this gene family is involved in a wide range of biological processes, including seed germination, hypocotyl elongation, photoperiod response, heading period regulation, senescence, fertility, and stress resistance. These factors exhibit rich and diverse biological functions. However, it has not yet been observed that genes containing the FHY3 domain promote the development of primary and secondary branches in rice panicles, increase the number of panicles and spikelets, or improve rice yield.

### SUMMARY

In the present invention, the Oryza longistaminata large-panicle introgression line material 1762 (Long et al., Crop Journal, 2023, 11:1541-1549) is hybridized and backcrossed with the Oryza longistaminata introgression line receptor parent 9311 to create a near-isogenic line. OlGn8.2, an important gene for controlling the grain number of panicles, is isolated using the map-based cloning method. This gene, specific to Oryza longistaminata, promotes rice growth, increases the primary and secondary branches, raises the grain number per panicles, and thus enhances rice yield. It can be widely used in the breeding of new high-yield rice varieties.

The objective of this invention is to provide the high-yield gene OlGn8.2 from Oryza longistaminata and, based on the relationship of this gene with the development of the primary and secondary branches of rice inflorescence, further provide the application of the OlGn8.2 gene in improving rice grain yield.

The objectives of this invention can be achieved through the following methods:

By introducing the OlGn8.2 gene into rice or expressing it through crossbreeding, the stem becomes thicker when the OlGn8.2 gene is introduced into the rice near-isogenic line NIL-OlGn8.2 or expressed in rice variety 9311. During maturity, the plants exhibit a significantly increased primary and secondary branches of panicles, as well as an increased grain number per panicle and enhanced yield at both the population and individual plant levels when compared to the 9311 variety. These findings suggest that OlGn8.2 has a direct role in enhancing rice growth and development, increasing the number of primary and secondary branches, and ultimately improving rice grain yield. OlGn8.2 is a gene specific to Oryza longistaminata that can function by interacting with the OsMADS18 gene in rice. In addition to sorghum and corn, the OsMADS18 protein is highly conserved in other food crops, which suggests that this gene may influence the yields of other monocotyledonous grass crops in similar molecular mechanism.

The Oryza longistaminata produces the protein OlGn8.2, which plays a role in regulating the development of the primary and secondary branches of rice panicles, increasing rice grain production, and improving rice yield. Its amino acid sequence is shown in SEQ ID NO.4.

To facilitate the research and utilization of the OlGn8.2 proteins, a tag as shown in Table 1 can be connected to either the amino or carboxyl terminus of the protein sequence.

TABLE 1
Tags and the corresponding amino acid sequences

| Tag types | Amino acid residues | Sequence |
|---|---|---|
| Poly-Arg | 5-6 (usually 5) | RRRRR (SEQ ID NO. 5) |
| Poly-His | 2-10 (usually 6) | HHHHHH (SEQ ID NO. 6) |
| FLAG | 8 | DYKDDDDK (SEQ ID NO. 7) |
| Strep-tag II | 8 | WSHPQFEK (SEQ ID NO. 8) |
| c-myc | 10 | EQKLISEEDL (SEQ ID NO. 9) |

An OlGn8.2 gene involved in regulating the development of primary and secondary branches in rice panicles, increasing the grain number per panicle, and improving rice yield, has a nucleotide sequence as shown in SEQ ID NO.1, 2, or 3.

SEQ ID NO.1 shows the genomic base sequence consisting of 2395 bases, including the promoter, 5′UTR, exons, and 3′UTR.

SEQ ID NO.2 shows the cDNA coding sequence.

SEQ ID NO.3 shows the CDS sequence.

A biological material related to the OlGn8.2 gene, which can be any of the following: primers for amplifying the OlGn8.2 gene, an expression cassette, a recombinant vector, a transgenic cell line, or recombinant bacteria containing the OlGn8.2 gene.

The OlGn8.2 gene is applied to promote the development of the primary and secondary branches in rice panicles, increase the grain number per panicle, and subsequently improve rice yield. In addition, primers for amplifying the entire OlGn8.2 gene, expression cassettes, recombinant vectors, transgenic cell lines, and recombinant bacteria containing the OlGn8.2 gene can be applied to rice to stimulate the development of primary and secondary branches, increase the grain number per panicle, and thus improve yields.

The OlGn8.2 gene can significantly increase the grain number per panicle and improve rice yield by increasing the number of primary and secondary branches, making it valuable for developing high-yield rice varieties.

Applications of primers for amplifying the full length of the OlGn8.2 gene in the development of high-yield rice varieties.

Applications of expression cassettes, recombinant vectors, transgenic cell lines, or recombinant bacteria containing the OlGn8.2 gene in the development of high-yield rice varieties.

Increasing the number of the primary and secondary branches in rice panicles, as well as the grain number per panicle and overall rice yield, can be achieved by either introducing the OlGn8.2 into cultivated rice varieties through crossbreeding or by transferring the OlGn8.2 into cultivated rice to ensure its normal or overexpression through genetic modification. Specifically, the OlGn8.2 gene can be introduced into other rice varieties by crossbreeding Oryza longistaminata or NIL-OlGn8.2 carrying the OlGn8.2 with other rice varieties and then using molecular marker-assisted selection to incorporate it into the other rice varieties. Alternatively, the OlGn8.2 can be overexpressed by transferring it into rice using an expression vector.

To construct a recombinant vector containing the OlGn8.2, existing crop transformation vectors can be selected. These crop transformation vectors include binary Agrobacterium vectors and vectors suitable for microprojectile bombardment in crops, such as pCAMBIA3301, pYLCRISPR/Cas9Pubi-B, pYLCRISPR/Cas9P35S-H, PYLCRISPR/Cas9P35S-N, pCAMBIA2301, pH7WG2D, or other vectors related to editing technologies like TALENs and ZFNs.

In order to increase rice yield by using the OlGn8.2 gene, any promoter that affects the expression of the OlGn8.2 gene can be added before the gene's start site during the vector construction. Promoters such as the cauliflower mosaic virus (CaMV) 35S promoter or the ubiquitin gene promoter (pUbi) can be used. Additionally, enhancers can be added to boost expression. Regardless of the method used, it is crucial to ensure the accuracy of the coding sequence to obtain the correct OlGn8.2 protein structure.

Recombinant vectors that include marker genes can be used to construct the desired recombinant vectors. Examples of such marker genes include GUS, GFP, genes conferring hygromycin resistance, and genes conferring herbicide resistance. Incorporating these marker genes into the recombinant vectors makes the experimental process more convenient and facilitates the subsequent screening and identification of transformed organisms.

The recombinant vector containing OlGn8.2 can be introduced into crop tissues or cells by various methods, including microinjection, Agrobacterium-mediated genetic transformation, or common techniques using Ti plasmid, Ri plasmid, or viral vectors.

Additionally, the OlGn8.2 gene itself can serve as a molecular marker in rice breeding.

The above-mentioned rice includes indica rice, japonica rice, etc.

This invention promotes the development of the primary and secondary branches in rice panicles by introducing the OlGn8.2 through crossbreeding between rice varieties or by transgenic techniques. This demonstrates that the gene can be used to enhance rice growth and development, increase the primary and secondary branch number, and be applied in the breeding of high-yield rice crops. Therefore, the OlGn8.2 gene provides a powerful tool and method for molecular marker-assisted breeding and genetic engineering to cultivate new high-yield rice varieties, offering significant application potential.

### BRIEF DESCRIPTION OF THE DRAWINGS

FIG. 1A and FIG. 1B: shows the morphological character of the Oryza longistaminata introgression line 1762. FIG. 1A, plant and panicle type of introgression line 1762. FIG. 1B, statistical results of the primary branches and secondary branch number of introgression line 1762.

FIG. 2 shows the plant and panicle types of the OlGn8.2 near-isogenic lines (NIL-OlGn8.2).

FIG. 3 shows the structure of the OlGn8.2 gene, which contains the FHY3 domain.

FIG. 4 shows the schematic diagram of the OlGn8.2 gene overexpression vector structure.

FIG. 5 shows the modeled structure of the protein encoded by the OlGn8.2 gene.

FIG. 6 shows the molecular detection of OlGn8.2 in OlGn8.2 gene overexpression transgenic plants (OlGn8.2-OE).

FIG. 7 shows the detection of OlGn8.2 expression levels in NIL-OlGn8.2 and OlGn8.2 transgenic lines (OlGn8.2-OE).

FIG. 8 shows the plant and panicle type of the OlGn8.2 overexpression transgenic line (OlGn8.2-OE).

### DESCRIPTION OF THE EMBODIMENTS

This section shows how OlGn8.2 gene can be used specifically to increase the primary and secondary branch number and therefore increase rice yield by enhancing primary and secondary branching, and grain number. However, the invention is not limited to the following implementation examples.

In the following example, the rice is managed according to the conventional cultivation method: Firstly, fresh rice seeds are soaked for germination, and sown in a prepared nursery. When the seedlings reach the 4-leaf stage, they are transplanted to the paddy field, and then the yield-related phenotypes are examined at the maturity stage.

### Example 1: Creation of OlGn8.2 High-Yielding Near Isogenic Line (NIL-OlGn8.2) in the Background of 9311

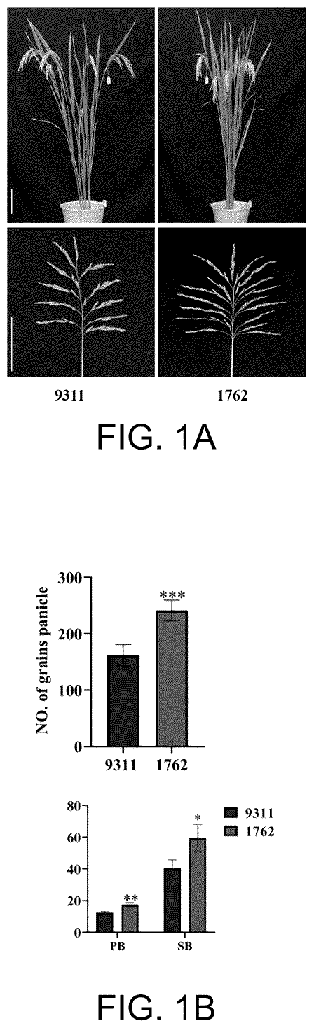

1. The F₁ was obtained by crossing Oryza longistaminata with an excellent indica rice variety, 9311. The BC₂F₁ was obtained after backcrossing with 9311 twice, and then the population was self-pollinated for 20 generations to obtain a stable, homozygous introgression line population containing chromosome fragments from the Oryza longistaminata. In this introgression line population, the primary and secondary branch number and the panicle size of the introgression line 1762 were significantly increased compared to the recipient parent 9311 (see FIG. 1A and FIG. 1B). Therefore, the Oryza longistaminata introgression line 1762 was selected as the candidate material for cloning the high-yield gene OlGn8.2.

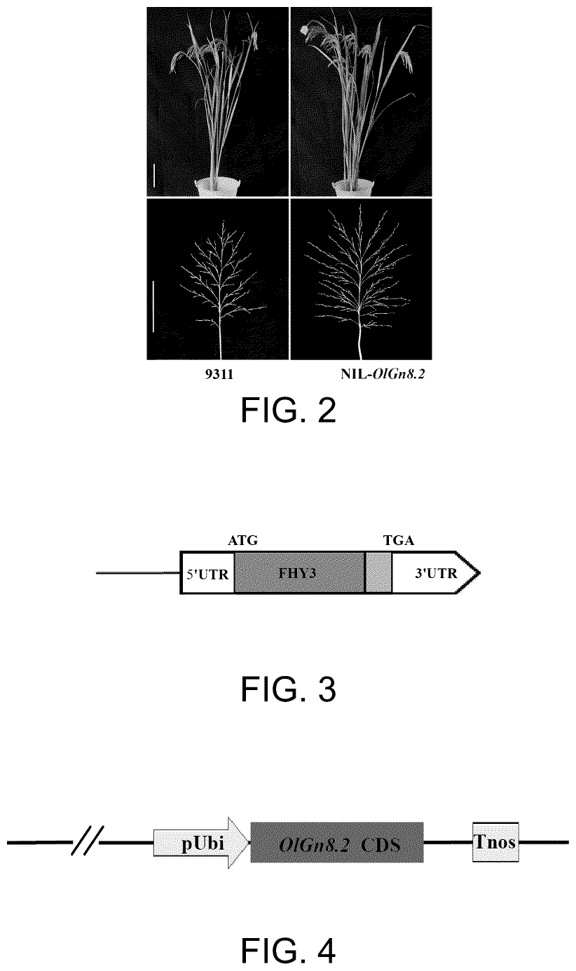

2. Construction of OlGn8.2 Near-Isogenic Line (NIL-OlGn8.2): To clone the large panicle gene from line 1762, it was crossed with 9311 and then backcrossed for 3 generations. The resulting population was self-pollinated, and a single plant was identified that had a plant type almost identical to 9311, but with a significantly increased number of primary and secondary branches and larger panicle size. At the genomic level, this plant was identical to 9311 except for a small DNA fragment from the Oryza longistaminata on the long arm of chromosome 8. Gene cloning showed that this region contained the large panicle gene OlGn8.2 from the Oryza longistaminata, so this line was designated as the OlGn8.2 near-isogenic line (NIL-OlGn8.2). Compared to 9311, the panicles of NIL-OlGn8.2 were larger, and the primary and secondary branch number, and grain number was significantly increased (see FIG. 2). The agronomic characters of NIL-OlGn8.2 showed no significant differences from 9311 in 1000-grain weight, seed setting rate, and effective panicle number. However, the primary and secondary branch number and grains per panicle increased by 4.87, 16.34, and 109.40, respectively, compared to 9311. The yield per plant of NIL-OlGn8.2 also increased by 36.12% compared to 9311 (see Table 2).

TABLE 2
Yield traits of 9311 and NIL-OlGn8.2

| Agronomic Traits | 9311 | NIL-OlGn8.2 |
|---|---|---|
| Number of Primary Branches | 11.84 ± 0.59 | 16.71 ± 1.76* |
| Number of Secondary Branches | 33.63 ± 1.19 | 49.70 ± 5.92* |
| Number of Grains per Panicle | 171.74 ± 17.59 | 281.14 ± 32.83 * |
| Seed Setting Rate (%) | 61.45 ± 4.75 | 69.78 ± 6.83 |
| Thousand Grain Weight (g) | 23.17 ± 1.48 | 24.66 ± 0.76 |
| Effective Panicles | 9.20 ± 1.46 | 7.40 ± 0.73 |
| Yield per Plant (g) | 21.88 ± 0.96 | 36.17 ± 4.84* |

### Example 2: Creation of High-Yielding Transgenic Lines of OlGn8.2

### 1. Obtaining the Full-Length Fragment of the OlGn8.2 Gene

According to the paper published by Long et al., in Crop Journal in 2023 (volume 11, pages 1541-1549), the cDNA from line 1762 was used as a template to design the primers OlGn8.2-F/R (the primer sequences are shown in Table 3). PCR amplification was performed using these primers. The nucleotide sequence of the amplified gene fragment is shown in SEQ ID NO.3. The structural diagram of the OlGn8.2 gene is shown in FIG. 3.

TABLE 3
Primer sequences

| Primer Name | Primer Sequence (5′-3′) |
|---|---|
| OlGn8.2-F | ATGCACCAAAAACATCCGAA |
| | (SEQ ID NO. 10) |
| OlGn8.2-R | CTAATAATCTGACTCAAATG |
| | (SEQ ID NO. 11) |

### 2. Construction of the OlGn8.2 Gene Overexpression Vector

The product amplified using the OlGn8.2-F/R primers was inserted into the expression vector pCAMBIA1301 containing the Ubiquitin promoter (strong promoter) through a recombination reaction. Positive clones were screened using the marker genes on the vector to obtain the recombinant expression vector OlGn8.2-OE.

### 3. Acquisition of the OlGn8.2 Gene Overexpression Transgenic Plants

The constructed OlGn8.2-OE transgenic vector can be introduced into Agrobacterium tumefaciens EHA105 via electroporation or heat shock methods. Subsequently, positive strains of Agrobacterium tumefaciens were selected and injected into rice tissue.

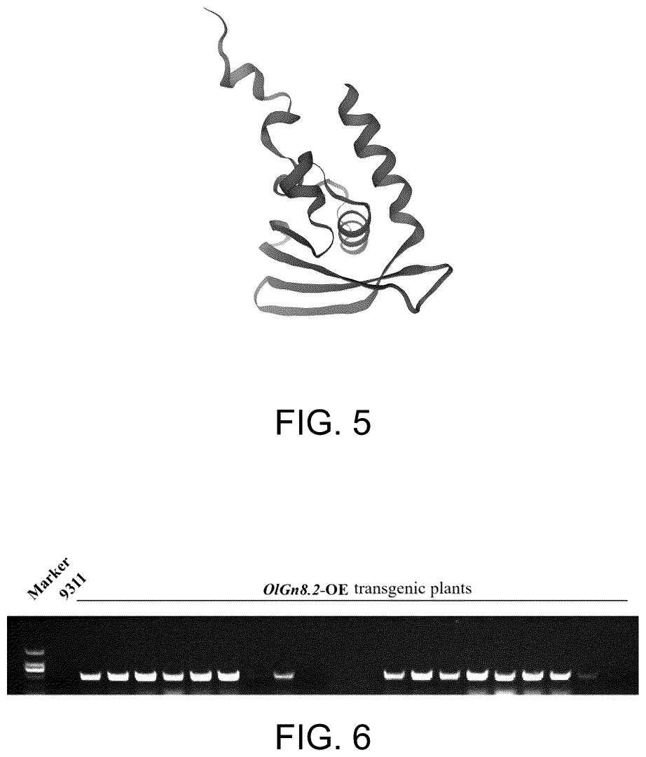

A recombinant Agrobacterium tumefaciens strain containing the OlGn8.2-OE plasmid was used to infect the callus of the rice cultivar 9311. A positive transgenic callus was selected on a culture medium containing 50 mg/L hygromycin. The positive callus was then differentiated, rooted, and transplanted to obtain the T₀ generation of transgenic plants. The T₁ generation plants were obtained by conventional breeding and molecular characterization methods. The model structure of the OlGn8.2 gene in the overexpression rice lines is shown in FIG. 4, and the CDS sequence of OlGn8.2 is provided in SEQ ID NO.3. The amino acid sequence of the protein encoded by the OlGn8.2 gene is shown in SEQ ID NO.4, and the modeled structure of the protein is presented in FIG. 5.

### 4. Detection of OlGn8.2 Gene in the Overexpression Transgenic Plants

### (1) PCR Detection of OlGn8.2 Gene Positive Strains

The genomic DNA of OlGn8.2 overexpression plants and wild-type plants (9311) was obtained using the conventional CTAB (cetyltrimethylammonium bromide) method to extract genomic DNA. Primers were designed, including the forward primer (pCAMBIA1301-F) targeting the pCAMBIA1301 overexpression vector and the OlGn8.2-specific reverse primer (OlGn8.20E-R) (the primer sequences are shown in Table 4). The genomic DNA of the wild-type plants was used as the negative control, and the genomic DNA of the OlGn8.2 overexpression plants and wild-type plants was amplified. All the OlGn8.2 overexpression transgenic plants with OlGn8.2 amplification bands were positive, as shown in FIG. 6.

TABLE 4
Primer sequences for positive identification of OlGn8.2
overexpression plants

| Primer Name | Primer Sequence (5′-3′) |
|---|---|
| pCAMBIA1301-F | CTGATGCATATACATGATGG (SEQ ID NO. 11) |
| OlGn8.20E-R | CTTCTTCCTTAGCTCGTACA (SEQ ID NO. 13) |

(2) qRT-PCR Detection of OlGn8.2 Gene Expression Levels

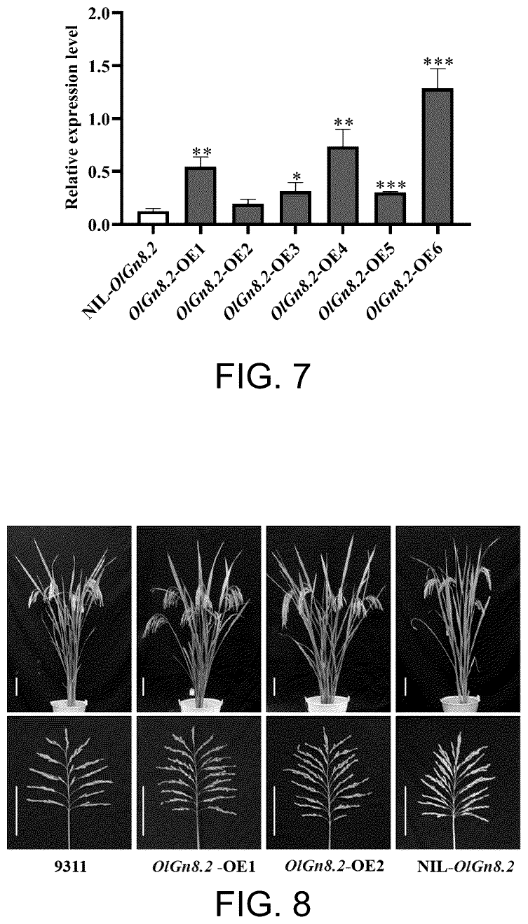

The ubiquitin promoter is a strong promoter in monocot plants, which can enhance the expression of the target gene in plants. The total RNA of the OlGn8.2 overexpressing plants and the wild-type 9311 plants was obtained using the conventional RNA extraction method, and the corresponding cDNA was synthesized using the reverse transcription kit (purchased from Invitrogen). The Actin gene was used as an internal reference, and the OlGn8.2 RT-F/R primers (primer sequences shown in Table 5) were used to perform qRT-PCR to detect the expression level of OlGn8.2 gene. The results showed that the expression level of the OlGn8.2 gene was significantly increased in the overexpression transgenic plants, as shown in FIG. 7.

TABLE 5
Primer sequences for detection of OlGn8.2 expression levels

| Primer Name | Primer Sequence (5′-3′) |
|---|---|
| OlGn8.2 RT-F | AGGTTATGCCCAATACTGAT (SEQ ID NO. 14) |
| OlGn8.2 RT-R | TCTCAAAGTCATCAACATCC (SEQ ID NO. 15) |
| Actin RT-F | AGCATGAAGATCAAGGTGGTC (SEQ ID NO. 16) |
| Actin RT-R | GCCTTGGCAATCCACATC (SEQ ID NO. 17) |

### (3) Observation and Investigation of Grain Number Per Panicle, Primary and Secondary Branches in Transgenic Plants

When the rice plants reached maturity, the seeds from the transgenic lines overexpressing OlGn8.2 and the wild-type 9311 control were collected separately. Observations of primary and secondary branches, grains per panicle, yield per plant, and weight of 1000 grains were made and analyzed statistically. The results showed that the transgenic plants expressing the OlGn8.2 gene had a strong plant architecture and a significantly larger panicle size compared to the wild-type 9311 (FIG. 8). The statistical analysis revealed that these agronomic traits were improved considerably in the over-expressing transgenic material compared to the wild-type 9311 (Table 6). In conclusion, the overexpression of the OlGn8.2 led to enhanced plant architecture and yield-related characteristics in the transgenic rice lines compared to the non-transgenic 9311 Variety.

TABLE 6
Agronomic traits of OlGn8.2 overexpression plants

| | | pUbi::OlGn8.2 | | |
|---|---|---|---|---|
| Agronomic Traits | WT (9311) | OlGn8.2-OE1 | OlGn8.2-OE2 | OlGn8.2-OE3 |
| Number of primary | 11.77 ± 0.71 | 14.64 ± 0.51** | 17.43 ± 1.81* | 19.29 ± 1.53** |
| branches | | | | |
| Number of secondary | 29.03 ± 2.61 | 44.87 ± 2.91** | 50.71 ± 3.91 ** | 50.13 ± 6.53* |
| branches | | | | |
| Number of grains per | 143.30 ± 15.84 | 231.13 ± 25.65* | 255.71 ± 30.68* | 265.55 ± 12.18*** |
| panicle | | | | |
| Seed setting rate (%) | 70.76 ± 5.78 | 68.78 ± 4.95 | 72.30 ± 2.34 | 72.47 ± 5.57 |
| Thousand grain | 23.20 ± 1.09 | 22.40 ± 1.60 | 21.47 ± 0.82 | 23.63 ± 0.85 |
| weight (g) | | | | |
| Effective panicles | 9.67 ± 1.25 | 8.33 ± 1.70 | 9.33 ± 0.47 | 8.00 ± 1.63 |
| Yield per plant (g) | 21.34 ± 2.23 | 26.70 ± 0.64* | 37.62 ± 0.76 *** | 34.30 ± 3.71*** |

*, **, ***indicate significant differences at P < 0.05, 0.01 and 0.001 levels, respectively.

It should be noted that the above examples are just one way of implementing the invention. There are no limitations to this invention beyond those mentioned above; any other implementations that have been modified, edited, rearranged, or changed based on the principles and spirit of this invention are considered equivalent substitutions and are also protected by this patent.

What is claimed is:

1. A high-yield and large panicle gene OlGn8.2 gene from wild rice Oryza longistaminata, wherein an amino acid sequence of a protein encoded by the OlGn8.2 gene is as shown in SEQ ID NO.4.

2. The OlGn8.2 gene according to claim 1, wherein a nucleotide sequence of the OlGn8.2 gene is any one of the sequences shown in SEQ ID NO.1, 2, or 3.

3. A biological material related to the OlGn8.2 gene according to claim 1, which is any one of following materials:
primers for amplifying the OlGn8.2 gene, expression cassettes, recombinant vectors, transgenic cell lines, or recombinant microorganisms containing the OlGn8.2 gene.

4. An application of OlGn8.2 gene according to claim 1, including at least one of following applications:
applications for increasing a number of primary and secondary rice branches, increasing a number of rice grains per panicle, increasing rice yield, and cultivating high-yield rice varieties.

5. An application of primers used to amplify the OlGn8.2 gene according to claim 1, including at least one of following applications:
applications for increasing a number of primary and secondary rice branches, increasing a number of rice grains per panicle, increasing rice yield, and cultivating high-yield rice varieties.

6. An application of an expression cassette, a recombinant vector, a transgenic cell line, or a recombinant microorganism containing the OlGn8.2 gene according to claim 1, including at least one of following applications:
applications for increasing a number of primary and secondary rice branches, increasing a number of rice grains per panicle, increasing rice yield, and cultivating high-yield rice varieties.

7. The application according to claim 4, wherein the rice includes indica rice or japonica rice.

8. The application according to claim 5, wherein the rice includes indica rice or japonica rice.

9. The application according to claim 6, wherein the rice includes indica rice or japonica rice.

10. The application according to claim 4, wherein the application is achieved by introducing the OlGn8.2 gene through hybridization transfer, or increasing an expression level of OlGn8.2 gene through gene transfer.

11. The application according to claim 5, wherein the application is achieved by introducing the OlGn8.2 gene through hybridization transfer, or increasing an expression level of OlGn8.2 gene through gene transfer.

12. The application according to claim 6, wherein the application is achieved by introducing the OlGn8.2 gene through hybridization transfer, or increasing an expression level of OlGn8.2 gene through gene transfer.

13. A method for increasing rice yield, including introducing the OlGn8.2 gene of claim 1 into rice through hybridization transfer, or transferring an expression vector into rice to overexpress the OlGn8.2 gene of claim 1.

14. An application of the OlGn8.2 gene of claim 1 as a molecular marker in rice breeding.


SEQUENCE LISTING (17 sequences)

SEQ ID NO: 1 - DNA, 2947 bp, Oryza sativa
  source 1..2947: mol_type = genomic DNA
ctccagccttagcctattctctcccgcatctgacctcccacagcttgtgccttgcccaaaccacggcggtgagagtgcactcatcgagccggcagcctccccacgcctcccatctccgtcctggttgccctgcctgccctgcagccactggagtggtcctggtaagacgctgacctaaatttggtactattgtattgattagtttatgcaatggttgttttgttctgcaatttttgttgtgcttaaccttaatgtcgacatgaaaatttactatctcattatgaaaaagaggtgagattttatcttgttgattgactttgttttggacgatctgccttagaacactaggttgtatttgacgagacaagtaggatgaaaatctgcattctgcaactagaatcgcattaatcctaggtatatagctagatgagtgtgctatgtaaaattaatgtaacagaggtgcaaattttgcaacttaaatttggtttaaattaaggtattatccatatatgtaataaagctggttccatcgaacttttatatcacaggttatcataaatcatttgttttaatatttgccaacagtctatcaagcctttattactttgtaatttgcaggatgtttttcgcaaagttatatcacttttatcattattcttacatgcaattttcgtattgtgttgtgatctgtgtgtgaaaaatctcatctttgtagacacttgatttgtgtgaagacttgtaaatttgtaaagatttttgttagtgatgtatgaaacgcaagatgttaacctattttttaattatgctgatatggttcttctctttgtgtatttttactgcataataacagtgttacacaatggacgaggacgaagttagcttggaggagatggaggagtataacatggttgctagtaagctttttagaagcgaggaagatggctacaagttttacaatgaatatgcaaggtgtaaaggttttagtattagaagggacaaagtgaagcgatttagtggtaccgagctactgttctggaggcgattttattgctcatgtgaaggttacaggacgttgaagaactttgagagaaccaataggaaaagggagccacgtgcgttgacccactgtgggtgtaaggcaatgcttgaaattgagctcaatggagagaccggaatgtggtttgtatcaggttttgaagctagacattcacatagacttgctaaccctgacctggtagctttcttaagatctcatcgtgaggtaaatgatgctcaaaaagctgaggccgtggagcttggagttggcgggcttcgaacatgtcaaataatggaagtaatggagaagaatcatggtgggtatgataaggttgggttcgtaatgcgagacttgtacaatttttttgcccggtacaagaagaagagaattaaaggtcgggacgctgatctcgtgatgaaccatctgatggctcagcaagaacaagacccagattttttctttagatacagtatagatgaaaagggtcgtctgaggaatttattttgggccgacagtcaatcccagcttgactatgaagcattcggtgacgtggtaatatttgatagtacataccgtgtgaacccgtacaatcttccatttgttccttttgttggagctaaccatcatcgcagcacagttatttttgggtgtggtatattgtcaaatgagtctgtttcttcatatgtctggctgctgcaaactcttttggaggcaatgcaccaaaaacatccgaaatcattgatcactgataaggatgcatcaatggcgaaggcaattacaaaggttatgcccaatactgatcatagattgtgtagttggcacattgaggagaacatgaaatgtcacctccgtcgccaaaagcttgcagattttaagaaattcctttatgatgctatggatgttgatgactttgagagatgttgggttgagtataaggccaagtatggattcaatgaaaataacttatggataagcatgatgtacgagctaaggaagaagtggtccactgcatatatgaaagggaaacgcttccttggaatgcggagcaaccaacggagcgagtctttgaactcaaggttgcataggcatcttgatcgaaagatgtcgctggtcgatttggttgagcactatgagttttgcttgtcacgtatccgtaggaatgaggccaagctggatgcgagagtgtcgcagtctgtctcgttcactacaatatgtgctgacccactagagaaaagtgtggcacgtatttacacgccggccatgttcaagaaggtctgggcagagataaggaagctatatgagtgggaagttttcaatgtggcaagacaagatggtgctggtgtgtttaccgttgcatcaaaagacaacaatgttgtgcaggttcatgtgaggtgtacttttgaggagcaatcgatgaatagtgccaattgcgattgcaaaaagctggagtgtgacggcatcccttgctctcatgtttgtgctgttcttaagtttcttggtgtcggcactattcctcgttgttgtgttatggtaaggtggacaatggatgcgaaagcggcatttgagtcagattattagaagtaccaacactcatgtgtggtctgagcagatggatttataccgtgacctgagaaacatgagtagtcttgctttatttatagcctctaaatcatctgagaaggcggaaaaggtaagaaatttctttcggagtattatggaggaagactcggtgaaccccaataaagagtggccgtttggtcctttgccagcgcactattcagcatgtaaccaaccccacaggacgagagtccttgatcctgaggtgattacttctaaaggtgccccgtcaaagaagagattgaggccaatcagtgattcttggaatagaagaacctttgaatacctggaagc

SEQ ID NO: 2 - DNA, 1247 bp, Oryza sativa
  source 1..1247: mol_type = other DNA
atgagtctgtttcttcatatgtctggctgctgcaaactcttttggaggcaatgcaccaaaaacatccgaaatcattgatcactgataaggatgcatcaatggcgaaggcaattacaaaggttatgcccaatactgatcatagattgtgtagttggcacattgaggagaacatgaaatgtcacctccgtcgccaaaagcttgcagattttaagaaattcctttatgatgctatggatgttgatgactttgagagatgttgggttgagtataaggccaagtatggattcaatgaaaataacttatggataagcatgatgtacgagctaaggaagaagtggtccactgcatatatgaaagggaaacgcttccttggaatgcggagcaaccaacggagcgagtctttgaactcaaggttgcataggcatcttgatcgaaagatgtcgctggtcgatttggttgagcactatgagttttgcttgtcacgtatccgtaggaatgaggccaagctggatgcgagagtgtcgcagtctgtctcgttcactacaatatgtgctgacccactagagaaaagtgtggcacgtatttacacgccggccatgttcaagaaggtctgggcagagataaggaagctatatgagtgggaagttttcaatgtggcaagacaagatggtgctggtgtgtttaccgttgcatcaaaagacaacaatgttgtgcaggttcatgtgaggtgtacttttgaggagcaatcgatgaatagtgccaattgcgattgcaaaaagctggagtgtgacggcatcccttgctctcatgtttgtgctgttcttaagtttcttggtgtcggcactattcctcgttgttgtgttatggtaaggtggacaatggatgcgaaagcggcatttgagtcagattattagaagtaccaacactcatgtgtggtctgagcagatggatttataccgtgacctgagaaacatgagtagtcttgctttatttatagcctctaaatcatctgagaaggcggaaaaggtaagaaatttctttcggagtattatggaggaagactcggtgaaccccaataaagagtggccgtttggtcctttgccagcgcactattcagcatgtaaccaaccccacaggacgagagtccttgatcctgaggtgattacttctaaaggtgccccgtcaaagaagagattgaggccaatcagtgattcttggaatagaagaacctttgaatacctggaagc

SEQ ID NO: 3 - DNA, 864 bp, Oryza sativa
  source 1..864: mol_type = other DNA
atgcaccaaaaacatccgaaatcattgatcactgataaggatgcatcaatggcgaaggcaattacaaaggttatgcccaatactgatcatagattgtgtagttggcacattgaggagaacatgaaatgtcacctccgtcgccaaaagcttgcagattttaagaaattcctttatgatgctatggatgttgatgactttgagagatgttgggttgagtataaggccaagtatggattcaatgaaaataacttatggataagcatgatgtacgagctaaggaagaagtggtccactgcatatatgaaagggaaacgcttccttggaatgcggagcaaccaacggagcgagtctttgaactcaaggttgcataggcatcttgatcgaaagatgtcgctggtcgatttggttgagcactatgagttttgcttgtcacgtatccgtaggaatgaggccaagctggatgcgagagtgtcgcagtctgtctcgttcactacaatatgtgctgacccactagagaaaagtgtggcacgtatttacacgccggccatgttcaagaaggtctgggcagagataaggaagctatatgagtgggaagttttcaatgtggcaagacaagatggtgctggtgtgtttaccgttgcatcaaaagacaacaatgttgtgcaggttcatgtgaggtgtacttttgaggagcaatcgatgaatagtgccaattgcgattgcaaaaagctggagtgtgacggcatcccttgctctcatgtttgtgctgttcttaagtttcttggtgtcggcactattcctcgttgttgtgttatggtaaggtggacaatggatgcgaaagcggcatttgagtcagattattag

SEQ ID NO: 4 - protein, 287 aa, Oryza sativa
MHQKHPKSLITDKDASMAKAITKVMPNTDHRLCSWHIEENMKCHLRRQKLADFKKFLYDAMDVDDFERCWVEYKAKYGFNENNLWISMMYELRKKWSTAYMKGKRFLGMRSNQRSESLNSRLHRHLDRKMSLVDLVEHYEFCLSRIRRNEAKLDARVSQSVSFTTICADPLEKSVARIYTPAMFKKVWAEIRKLYEWEVFNVARQDGAGVFTVASKDNNVVQVHVRCTFEEQSMNSANCDCKKLECDGIPCSHVCAVLKFLGVGTIPRCCVMVRWTMDAKAAFESDY

SEQ ID NO: 5 - protein, 5 aa, synthetic construct
RRRRR

SEQ ID NO: 6 - protein, 6 aa, synthetic construct
HHHHHH

SEQ ID NO: 7 - protein, 8 aa, synthetic construct
DYKDDDDK

SEQ ID NO: 8 - protein, 8 aa, synthetic construct
WSHPQFEK

SEQ ID NO: 9 - protein, 10 aa, synthetic construct
EQKLISEEDL

SEQ ID NO: 10 - DNA, 20 bp, synthetic construct
  source 1..20: mol_type = other DNA
atgcaccaaaaacatccgaa

SEQ ID NO: 11 - DNA, 20 bp, synthetic construct
  source 1..20: mol_type = other DNA
ctaataatctgactcaaatg

SEQ ID NO: 12 - DNA, 20 bp, synthetic construct
  source 1..20: mol_type = other DNA
ctgatgcatatacatgatgg

SEQ ID NO: 13 - DNA, 20 bp, synthetic construct
  source 1..20: mol_type = other DNA
cttcttccttagctcgtaca

SEQ ID NO: 14 - DNA, 20 bp, synthetic construct
  source 1..20: mol_type = other DNA
aggttatgcccaatactgat

SEQ ID NO: 15 - DNA, 20 bp, synthetic construct
  source 1..20: mol_type = other DNA
tctcaaagtcatcaacatcc

SEQ ID NO: 16 - DNA, 21 bp, synthetic construct
  source 1..21: mol_type = other DNA
agcatgaagatcaaggtggtc

SEQ ID NO: 17 - DNA, 18 bp, synthetic construct
  source 1..18: mol_type = other DNA
gccttggcaatccacatc